(19) United States
(12) Patent Application Publication
(10) Pub. No.: US 2024/0309280 A1
(43) Pub. Date: Sep. 19, 2024

(54) INTEGRATED PROCESSES AND SYSTEMS FOR PRODUCING PARA-XYLENES

(71) Applicant: Saudi Arabian Oil Company, Dhahran (SA)

(72) Inventors: Mohammed I. Alamer, Dhahran (SA); Ali S. Abdali, Al-Khobar (SA); Qi Xu, Dhahran (SA); Mosab T. Kheyami, Dhahran (SA)

(73) Assignee: Saudi Arabian Oil Company, Dhahran (SA)

(21) Appl. No.: 18/183,299

(22) Filed: Mar. 14, 2023

(51) Int. Cl. C10G 61/04; C10G 61/10

(52) U.S. Cl. CPC C10G 61/04; C10G 61/10; C10G 2300/70; C10G 2400/30

(57) ABSTRACT

An integrated process for producing para-xylenes may include catalytically reforming a naphtha feed stream to form a reformate stream; separating the reformate stream into a C₁-C₇ hydrocarbon stream and a C₈₊ hydrocarbon stream; exposing the C₁-C₇ hydrocarbon stream to a first solvent in a solvent extraction unit to form a non-aromatic hydrocarbon stream and a C₆-C₇ aromatics stream; separating the C₆-C₇ aromatics stream into at least a toluene feed stream; separating the C₈₊ hydrocarbon stream into a C₉₊ hydrocarbon stream and a xylene stream; separating the xylene stream in a p-xylene separation unit to form the para-xylene stream and a xylene isomer stream; isomerizing the xylene isomer stream to produce a para-xylene rich stream; and upgrading the toluene feed stream and the C₉₊ hydrocarbon stream in a hybrid dealkylation/transalkylation unit with a hydrogen stream and a hybrid transalkylation/dealkylation catalyst to produce a product stream including para-xylenes.

### TECHNICAL FIELD

Embodiments of the present disclosure generally relate to refining and upgrading hydrocarbon oil, and pertain particularly to an integrated process and system for upgrading a naphtha stream into para-xylenes.

### BACKGROUND

Aromatic compounds, such as benzene, toluene, and xylenes (BTX), are basic intermediates for many petrochemical industries. Particularly desired are para-xylenes, a xylene isomer of the group 1,2-dimethylbenzene, (ortho-xylene or o-xylene), 1,3-dimethylbenzene (meta-xylene or m-xylene), or 1,4-dimethylbenzene (para-xylene or p-xylene). Para-xylenes may be used as a raw material in the synthesis of commercial plastics in clothing fibers, liquid and food storage containers, and thermoforming for manufacturing, among other uses. Accordingly, methods of converting entire hydrocarbon feeds into para-xylenes are desired.

### SUMMARY

In BTX-generating refinery processes, a naphtha feed is first sent to a catalytic reformer. The catalytic reformer reforms the naphtha stream into a reformate stream that is aromatics-rich. The reformate stream is then typically separated into light (carbon content less than 8) and heavy (carbon content greater than 7) streams.

However, aromatic compounds with carbon contents greater than 9 (C₁₀₊) may ordinarily be purged from the heavy stream and not utilized in any manner, due to these streams being thought of as petroleum coke precursors, potentially deactivating catalysts, as well as building up as unconverted fractions in recycling streams. As hydrocarbon streams, and the subset of naphtha streams, may contain a large percentage of these C₁₀₊ fractions, considerable amounts of p-xylene and other xylene isomers are not capitalized on within existing processes. Accordingly, methods are desired for converting C₁₀₊ fractions in naphtha streams, so as to utilize the entire naphtha stream and maximize para-xylene yields.

Consequently, described herein are integrated processes and systems for producing para-xylenes from a naphtha feed stream, while providing the aforementioned benefits. Particularly, C₉₊ fractions, such as the C₁₀₊ fractions previously mentioned, may be converted utilizing a hybrid transalkylation/dealkylation unit and catalyst, resulting in increased xylene and para-xylene yield. In this way, the entire heavy fraction of the naphtha feed stream may be converted to xylene, and in particular, para-xylene.

In accordance with one embodiment herein, an integrated process for producing para-xylenes may include catalytically reforming a naphtha feed stream to form a reformate stream; separating the reformate stream into a C₁-C₇ hydrocarbon stream and a C₈₊ hydrocarbon stream; exposing the C₁-C₇ hydrocarbon stream to a first solvent in a solvent extraction unit to form a non-aromatic hydrocarbon stream and a C₆-C₇ aromatics stream; separating the C₆-C₇ aromatics stream into at least a toluene feed stream; separating the C₈₊ hydrocarbon stream into a C₉₊ hydrocarbon stream and a xylene stream including ortho-xylene, meta-xylene, and para-xylene; separating the xylene stream in a p-xylene separation unit to form the para-xylene stream and a xylene isomer stream including ortho-xylene and meta-xylene; isomerizing the xylene isomer stream with a isomerization catalyst to produce a para-xylene rich stream; and upgrading the toluene feed stream and the C₉₊ hydrocarbon stream in a hybrid dealkylation/transalkylation unit with a hydrogen stream and a hybrid transalkylation/dealkylation catalyst to produce a product stream including para-xylenes, wherein a ratio by weight of the toluene feed stream to the C₉₊ hydrocarbon stream is from 0.3 to 3.

In accordance with another embodiment herein, an integrated system for producing para-xylenes may include a catalytic reformer including a reforming catalyst; a first separator fluidly connected to the catalytic reformer and downstream from the catalytic reformer; a solvent extraction unit including a first solvent, the solvent extraction unit fluidly connected to and downstream from the first separator; a toluene separation unit fluidly connected to and downstream from the solvent extraction unit; a xylene separation unit fluidly connected to and downstream from the first separator; a hybrid transalkylation/dealkylation unit including a hybrid transalkylation/dealkylation catalyst, the hybrid transalkylation/dealkylation unit fluidly connected to and downstream from the toluene separation unit and the xylene separation unit; a p-xylene separation unit fluidly connected to and downstream from the xylene separation unit; and a xylene isomerization unit including a isomerization catalyst, the xylene isomerization unit fluidly connected to, downstream from, and upstream from the p-xylene separation unit.

Additional features and advantages of the embodiments described herein will be set forth in the detailed description which follows, and in part will be readily apparent to those skilled in the art from that description or recognized by practicing the embodiments described, including the detailed description and the claims which are provided infra.

### BRIEF DESCRIPTION OF THE DRAWINGS

The following detailed description of specific embodiments of the present disclosure can be best understood when read in conjunction with the following drawings in which:

Figure (FIG. 1 illustrates a process flow diagram for an exemplary process in accordance with embodiments described herein;

FIG. 2 illustrates another process flow diagram for an exemplary process in accordance with embodiments described herein; and

FIG. 3 illustrates comparative results of two pilot plant experiments for an exemplary process in accordance with embodiments described herein.

For the purpose of describing the simplified schematic illustrations and descriptions of the relevant figures, the numerous valves, temperature sensors, electronic controllers and the like that may be employed and well known to those of ordinary skill in the art of certain chemical processing operations are not included. Further, accompanying components that are often included in typical chemical processing operations, such as air supplies, catalyst hoppers, and flue gas handling systems, are not depicted. Accompanying components that are in hydrotreating units, such as bleed streams, spent catalyst discharge subsystems, and catalyst replacement sub-systems are also not shown. It should be understood that these components are within the spirit and scope of the present embodiments disclosed. However, operational components, such as those described in the present disclosure, may be added to the embodiments described in this disclosure.

It should further be noted that arrows in the drawings refer to process streams. However, the arrows may equivalently refer to transfer lines, which may serve to transfer process streams between two or more system components. Additionally, arrows that connect to system components define inlets or outlets in each given system component. The arrow direction corresponds generally with the major direction of movement of the materials of the stream contained within the physical transfer line signified by the arrow. Furthermore, arrows, which do not connect two or more system components, signify a product stream, which exits the depicted system, or a system inlet stream, which enters the depicted system. Product streams may be further processed in accompanying chemical processing systems or may be commercialized as end products. System inlet streams may be streams transferred from accompanying chemical processing systems or may be non-processed feedstock streams. Some arrows may represent recycle streams, which are effluent streams of system components that are recycled back into the system. However, it should be understood that any represented recycle stream, in some embodiments, may be replaced by a system inlet stream of the same material, and that a portion of a recycle stream may exit the system as a product.

Additionally, arrows in the drawings may schematically depict process steps of transporting a stream from one system component to another system component. For example, an arrow from one system component pointing to another system component may represent “passing” a system component effluent to another system component, which may include the contents of a process stream “exiting” or being “removed” from one system component and “introducing” the contents of that product stream to another system component.

It should be understood that according to the embodiments presented in the relevant figures, an arrow between two system components may signify that the stream is not processed between the two system components. In other embodiments, the stream signified by the arrow may have substantially the same composition throughout its transport between the two system components. Additionally, it should be understood that in embodiments, an arrow may represent that at least 75 wt. %, at least 90 wt. %, at least 95 wt. %, at least 99 wt. %, at least 99.9 wt. %, or even 100 wt. % of the stream is transported between the system components. As such, in embodiments, less than all of the stream signified by an arrow may be transported between the system components, such as if a slip stream is present.

It should be understood that two or more process streams are “mixed” or “combined” when two or more lines intersect in the schematic flow diagrams of the relevant figures. Mixing or combining may also include mixing by directly introducing both streams into a like reactor, separation unit, or other system component. For example, it should be understood that when two streams are depicted as being combined directly prior to entering a separation unit or reactor, that in embodiments the streams could equivalently be introduced into the separation unit or reactor and be mixed in the reactor. Alternatively, when two streams are depicted to independently enter a system component, they may in embodiments be mixed together before entering that system component.

Reference will now be made in greater detail to various embodiments, some embodiments of which are illustrated in the accompanying drawings. Whenever possible, the same reference numerals will be used throughout the drawings to refer to the same or similar parts.

### DETAILED DESCRIPTION

Embodiments herein are directed to integrated systems and processes for forming para-xylenes from a naphtha stream, while providing the aforementioned benefits.

As used herein, the term “C_{#}hydrocarbons”, wherein “#” is a positive integer, is meant to describe all hydrocarbons having #carbon atoms. Moreover, the term “C++ hydrocarbons” is meant to describe primarily all hydrocarbon molecules having #or more carbon atoms. Accordingly, the term C₈₊ hydrocarbons” is meant to describe a mixture of hydrocarbons containing primarily 8 or more carbon atoms. Similarly, the term “C_{#}hydrocarbons” is meant to describe primarily all hydrocarbon molecules having #or less carbon atoms. Similarly, the term “C_{#}-C_{#′}” hydrocarbons is meant to describe a mixture of hydrocarbon molecules containing primarily between #and #′carbon atoms.

As used herein, a “catalyst” refers to any substance that increases the rate of a specific chemical reaction. Catalysts described in this disclosure may be utilized to promote various reactions, such as, but not limited to, cracking (including aromatic cracking), demetalization, desulfurization, denitrogenation, methylation, disproportionation, dealkylation, dearylation, transalkylation, and isomerization. As used herein, “cracking” generally refers to a chemical reaction where carbon-carbon bonds are broken. For example, a molecule having carbon to carbon bonds is broken into more than one molecule by the breaking of one or more of the carbon to carbon bonds, or is converted from a compound which includes a cyclic moiety, such as a cycloalkane, cycloalkane, naphthalene, an aromatic or the like, to a compound which does not include a cyclic moiety or contains fewer cyclic moieties than prior to cracking.

As used herein, “catalytic reforming” refers to a conversion process in petroleum refining and petrochemical industries. The reforming process generally catalytically converts low octane naphtha distilled from crude oil into higher octane reformate that contains aromatic compounds with a high amount of BTX. Generally, there are four major types of reactions taking place during reforming processes: (1) dehydrogenation of naphthenes to aromatics; (2) dehydrocyclization of paraffins to aromatics; (3) isomerization; and (4) hydrocracking.

As used herein, the term “crude oil” is to be understood to mean a mixture of petroleum liquids, gases, or combinations of liquids and gases, including some impurities such as sulfur-containing compounds, nitrogen-containing compounds and metal compounds that have not undergone significant separation or reaction processes. Crude oils are distinguished from fractions of crude oil. As used herein, the crude oil may be a minimally treated crude oil to provide a hydrocarbon oil feedstock having total metals (Nickel+Vanadium) content of less than 5 parts per million by weight (ppmw) and Conradson carbon residue of less than 5 wt. % Such minimally treated materials may be considered crude oils as described herein.

It should be understood that an “effluent” generally refers to a stream that exits a system component such as a separation unit, a reactor, or reaction zone, following a particular reaction or separation, and generally has a different composition (at least proportionally) than the stream that entered the separation unit, reactor, or reaction zone.

As used herein, the term “hydrogen/feed ratio” or “hydrogen-to-feed ratio,” or “hydrogen to feed stream ratio” refers to a standard measure of the volume rate of hydrogen circulating through the reactor with respect to the volume of feed. The hydrogen/feed ratio may be determined by comparing the flow volume of a hydrogen stream and the flow volume of a feed stream into a reactor.

As used herein, the term “naphtha” refers to a mixture of substances primarily including C₅ to C₁₁ hydrocarbons. “Light naphtha,” as used herein, is a fraction of naphtha primarily including C₅ to C₆ hydrocarbons, but which may also include C₇ hydrocarbons. As used herein, the term “heavy naphtha” refers to a fraction of naphtha primarily including C₇ to C₁₁ hydrocarbons.

As used herein, a “reactor” refers to a vessel in which one or more chemical reactions may occur between one or more reactants optionally in the presence of one or more catalysts. For example, a reactor may include a tank or tubular reactor configured to operate as a batch reactor, a continuous stirred-tank reactor (CSTR), or a plug flow reactor. Exemplary reactors include packed bed reactors such as fixed-bed reactors, and fluidized bed reactors. One or more “reaction zones” may be disposed in a reactor. As used herein, a “reaction zone” refers to an area where a particular reaction takes place in a reactor. For example, a packed bed reactor with multiple catalyst beds may have multiple reaction zones, where each reaction zone is defined by the area of each catalyst bed.

As used herein, any stream that is referred to as “rich” in some chemical species contains 50% or more by volume or weight of that chemical species, such as from 50% to 100%, or from 50% to 99%, the remaining 1% including trace amounts of other chemical species.

As used herein, a “separation unit” or “separator” refers to any separation device that at least partially separates one or more chemicals that are mixed in a process stream from one another. For example, a separation unit may selectively separate differing chemical species, phases, or sized material from one another, forming one or more chemical fractions. Examples of separation units include, without limitation, distillation columns, flash drums, knock-out drums, knock-out pots, centrifuges, cyclones, filtration devices, traps, scrubbers, expansion devices, membranes, solvent extraction devices, and the like. It should be understood that separation processes described in this disclosure may not completely separate all of one chemical constituent from all of another chemical constituent. It should be understood that the separation processes described in this disclosure “at least partially” separate different chemical components from one another, and that even if not explicitly stated, it should be understood that separation may include only partial separation. As used herein, one or more chemical constituents may be “separated” from a process stream to form a new process stream. Generally, a process stream may enter a separation unit and be divided, or separated, into two or more process streams of desired composition. Further, in some separation processes, a “lower boiling point fraction” (sometimes referred to as a “light fraction” or “light fraction stream”) and a “higher boiling point fraction” (sometimes referred to as a “heavy fraction,” “heavy hydrocarbon fraction,” or “heavy hydrocarbon fraction stream”) may exit the separation unit, where, on average, the contents of the lower boiling point fraction stream have a lower boiling point than the higher boiling point fraction stream. Other streams may fall between the lower boiling point fraction and the higher boiling point fraction, such as a “medium boiling point fraction.”

As used throughout this disclosure, “zeolites” may refer to micropore-containing inorganic materials with regular intra-crystalline cavities and channels of molecular dimension. Zeolites generally comprise a crystalline structure, as opposed to an amorphous structure such as what may be observed in some porous materials such as amorphous silica. Zeolites generally include a microporous framework, which may be identified by a framework type. The microporous structure of zeolites (e.g., 0.3 nm to 2 nm pore size) may render large surface areas and desirable size-/shape-selectivity, which may be advantageous for catalysis. The zeolites described may include, for example, aluminosilicates, titanosilicates, or pure silicates. In embodiments, the zeolites described may include micropores (present in the microstructure of a zeolite), and additionally include mesopores. As used throughout this disclosure, micropores refer to pores in a structure that have a diameter of greater than or equal to 0.1 nm and less than or equal to 2 nm, and mesopores refer to pores in a structure that have a diameter of greater than 2 nm and less than or equal to 50 nm. Unless otherwise described herein, the “pore size” of a material refers to the average pore size, but materials may additionally include micropores and/or mesopores having a particular size that is not identical to the average pore size.

It should further be understood that streams may be named for the components of the stream, and the component for which the stream is named may be the major component of the stream (such as comprising from 50 weight percent (wt. %), from 70 wt. %, from 90 wt. %, from 95 wt. %, from 99 wt. %, from 99.5 wt. %, or even from 99.9 wt. % of the contents of the stream to 100 wt. % of the contents of the stream). It should also be understood that components of a stream are disclosed as passing from one system component to another when a stream comprising that component is disclosed as passing from that system component to another. By way of non-limiting example, a referenced “C₂-C₄ hydrocarbon stream” passing from a first system component to a second system component should be understood to equivalently disclose “C₂-C₄ hydrocarbons” passing from a first system component to a second system component, and the like.

Referring initially to FIG. 1, an integrated system 100 for the conversion of naphtha feedstocks is illustrated. As used herein, “feedstock” may also be used to refer to “feed stock(s)” or “feed stream(s).” The integrated system 100 may include a catalytic reformer 102, a first separator 104, a solvent extraction unit 106, a toluene separation unit 107, a xylene separation unit 110, a hybrid transalkylation/dealkylation unit 116, a p-xylene separation unit 118, and a xylene isomerization unit 120. The toluene separation unit 107 may further include a benzene column 108 along with a toluene column 109, which may both be atmospheric distillation units, as explained in more detail herein.

The first separator 104 may be fluidly connected to both the catalytic reformer 102 and the solvent extraction unit 106, downstream from the catalytic reformer 102, and upstream from the solvent extraction unit 106. The benzene column 108 (which may also be regarded as a second separator 108) may be fluidly connected to the solvent extraction unit 106 and the toluene column 109, downstream from the solvent extraction unit 106, and upstream from the toluene column 109. The toluene column 109 (which may also be regarded as a third separator 109) may be fluidly connected to and upstream from the hybrid transalkylation/dealkylation unit 116. The xylene separation unit 110 (which may also be regarded as a fourth separator 110) may be fluidly connected to the first separator 104, the hybrid transalkylation/dealkylation unit 116, and the p-xylene separation unit 118. The xylene separation unit 110 may be downstream from the first separator 104, and upstream from the hybrid transalkylation/dealkylation unit 116 and the p-xylene separation unit 118.

The p-xylene separation unit 118 (which may also be regarded as a fifth separator) may be fluidly connected to the xylene separation unit 110 and the xylene isomerization unit 120. The p-xylene separation unit 118 may also be downstream from the xylene separation unit 110 and upstream from the xylene isomerization unit 120. The xylene isomerization unit 120 may in turn also be upstream of the p-xylene separation unit 118, i.e., the xylene isomerization unit 120 may recycle its products to the p-xylene separation unit 118.

Still referring to FIG. 1, the catalytic reformer 102 may catalytically reform a naphtha feed stream 2 utilizing a catalytic reformation catalyst to produce a reformate stream 4. The reformate stream 4 may be rich in aromatic compounds, particularly C₆₊ or C₆-C₁₂ aromatics. As previously described, any stream that is referred to as “rich” in some chemical species contains 50% or more by volume or weight of that chemical species, such as from 50% to 100%, or from 50% to 99%, the remaining 1% including trace amounts of other chemical species.

The catalytic reformer 102 may be operated at an operating temperature in the range of from 450° C. to 600° C. The catalytic reformer 102 may also be operated at an operating pressure in the range of from 0.3 MPa to 7 MPa. The catalytic reformer may also be operated with a liquid hourly space velocity of from 0.1 hr⁻¹ to 5 hr⁻¹.

The catalytic reformation catalyst may include a support and a precious metal, the support including a silica, alumina, or silica-alumina, and the precious metal including platinum, ruthenium, or both. The reforming catalyst may also be chlorided. The first separator 104 may receive the reformate stream 4 and may separate the same into a C₁-C₇ hydrocarbon stream 6 and a C₈₊ hydrocarbon stream 8.

The naphtha feed stream 2 may include a light naphtha or a heavy naphtha. The naphtha feed stream 2 may also include one or more non-hydrocarbon constituents, such as one or more heavy metals, sulfur compounds, nitrogen compounds, inorganic components, or other non-hydrocarbon compounds. The naphtha feed stream 2 may also be a hydrotreated naphtha, hydrotreated light naphtha, or a hydrotreated heavy naphtha, such that at least a portion of the one or more non-hydrocarbon constituents may be removed from the naphtha feed stream 2.

The solvent extraction unit 106 may receive the C₁-C₇ hydrocarbon stream 6 along with a first solvent 5. The solvent extraction unit 106 may thereby expose the C₁-C₇ hydrocarbon stream 6 to the first solvent 5, forming a non-aromatic hydrocarbon stream 10 and a C₆-C₇ aromatics stream 12. The non-aromatic hydrocarbon stream 10 may include C₁-C₇ non-aromatic hydrocarbon gases and hydrogen. The C₆-C₇ aromatics stream 12 may include toluene and benzene, and may be rich in toluene, benzene, or the combination thereof.

In embodiments, the solvent extraction unit 106 may include one or more extractive distillation columns, one or more absorbent columns, or combinations thereof. The first solvent 5 may include polar solvents, such as but not limited to sulfolane, n-methylpyrrolidone, di-methyl sulfoxide, n-formyl morpholine, polyglycol, or combinations thereof. The solvent extraction unit 106 may be operated at an operating temperature in the range of from 160° C. to 220° C. The solvent extraction unit 106 may also be operated at an operating pressure in the range of from 0.5 MPa to 20 MPa.

The toluene separation unit 107, particularly the benzene column 108, may receive the aromatics stream 12 and may thereby separate the C₆-C₇ aromatics stream 12 into a benzene-rich stream 13 and a toluene-rich stream 14. The toluene column 109 may receive the toluene-rich stream 14 and may form the toluene feed stream 15. The toluene separation unit 107 may also send the toluene feed stream 15 to the hybrid transalkylation/dealkylation unit 116, such as through the toluene column 109. The toluene feed stream 15 may also be combined with an external toluene stream where toluene within the system is not at a sufficient level for the desired transalkylation.

Still referring to FIG. 1, the xylene separation unit 110 may receive the C₈₊ hydrocarbon stream 8 and may separate the same into a C₉₊ hydrocarbon stream 18 and a xylene stream 20. The xylene stream may include ortho-xylene, meta-xylene, and para-xylene. The hybrid transalkylation/dealkylation unit 116 may then receive the C₉₊ hydrocarbon stream 18 and the toluene feed stream 15. The hybrid transalkylation/dealkylation unit 116 may then upgrade the combination of the two streams with a hydrogen stream 22 into a product stream 24, which may include xylene.

The product stream 24 may also include unconverted C₉ and C₁₀₊ hydrocarbon fractions, including but not limited to tri-methyl benzene (TMB), methyl-ethyl benzene (MEB), tetra-methyl benzene, di-methyl-ethyl benzene, tetra-ethyl benzene, as well as unconverted toluene fractions. In embodiments, the product stream may also include aromatic fractions produced from disproportionation of the toluene and C₉₊ hydrocarbon fractions. In one non-limiting example, toluene and tri-methyl benzene may form benzene and tetra-methyl benzene, respectively. Without being limited by theory, this may be due to the transalkylation reaction of tri-methyl benzene and toluene, among other C₉₊ aromatic-toluene pairs, being an equilibrium reaction, wherein at least some disproportionation of the involved chemical species occurs when exposed to the hybrid transalkylation/dcalkylation catalyst.

Additionally, without being limited by theory, the efficiency of the transalkylation reactions to produce xylene from TMB and toluene, among other C₉₊ aromatic-toluene pairs, may be impacted by the volumetric ratio of these species entering the hybrid transalkylation/dealkylation unit 116, at least because the TMB/toluene transalkylation reaction is an equilibrium reaction, as described above. Accordingly, it is contemplated that a ratio of the toluene feed stream 15 to the C₉₊ hydrocarbon stream 18 may be from 0.3 to 3 in embodiments herein. At ratios less than 0.3, insufficient toluene may be present within the hybrid transalkylation/dealkylation unit 116, and thereby less energy efficient disproportionation reactions, involving the C₉₊ hydrocarbons may dominate the hybrid transalkylation/dealkylation unit 116 over transalkylation reactions. At ratios above 3, the reverse may be the case, with the overabundant toluenes undergoing less energy efficient toluene disproportionation reactions. Without being limited be theory, the transalkylation of TMB and toluene may be regarded as more energy efficient due to the products of the reaction being entirely xylene, rather than xylene and a non-xylene aromatic.

The hybrid transalkylation/dealkylation catalyst may include a solid zeolite composite mixed with an alumina binder. The solid zeolite composite may have a large pore mordenite and a medium pore ZSM-5. The large pore mordenite and the medium pore ZSM-5 may be in a 1:1 to 5:1 weight ratio in the solid zeolite composite. Also as used herein, “large pore” zeolites are defined as zeolites with 12 membered rings forming the zeolite framework. Also as used herein, “medium pore” zeolites are defined as zeolites with 10 membered rings forming the zeolite framework.

Further, the hybrid transalkylation/dealkylation catalyst may have a mesostructure including at least one disordered mesophase and at least one ordered mesophase. The ordered mesophase may be a hexagonal mesophase, and the disordered mesophase may include a hexagonal mesophase. As used herein, “ordered mesophase” may refer to a crystalline zeolite uniform arrangement of mesopores, where “mesopores” have an average pore diameter between 2 and 50 nanometers. As used herein, “disordered mesophase” means a non-uniform arrangement of pores, where mesopores have an average pore diameter between 2 and 50 nanometers. The hybrid transalkylation/dealkylation catalyst may also include an active metal. The active metal may be impregnated on the hybrid transalkylation/dealkylation catalyst. The active metals may be selected from the group consisting of molybdenum, chromium, platinum, nickel, palladium, rhenium, or combinations thereof.

The hybrid transalkylation/dealkylation unit 116 may be operated at an operating temperature in the range of from 300° C. to 480° C. The hybrid transalkylation/dealkylation unit 116 may also be operated at an operating pressure in the range of from 1 MPa to 3 MPa. The hybrid transalkylation/dealkylation unit 116 may also be operated with a liquid hourly space velocity of from 0.1 hr⁻¹ to 10 hr⁻¹. The hybrid transalkylation/dealkylation unit 116 may also be operated with a hydrogen to feed stream (streams 15 and 18) ratio of from 1 to 6.

Still referring to FIG. 1, the system 100 may further include a sixth separator 122. The sixth separator 122 may be fluidly connected to and downstream from the hybrid transalkylation/dealkylation unit 116. The sixth separator 122 may also be fluidly connected to and upstream from the xylene separator 110, the benzene column 108, and the p-xylene separation unit 118. The sixth separator 122 may receive the product stream 24 and may separate the product stream 24 into at least additional non-aromatic hydrocarbon stream 10, additional C₆-C₇ aromatics stream 12, additional xylene stream 20, and an unconverted C₉₊ hydrocarbon stream 26. The unconverted C₉₊ hydrocarbon stream 26 may particularly include an unconverted C₉ hydrocarbon fraction stream and an unconverted C₁₀₊ hydrocarbon fraction stream. In embodiments, the sixth separator 122 may also send the unconverted C₉₊ hydrocarbon stream 26 to be combined with the C₉₊ hydrocarbon stream 26. Accordingly, the unconverted C₉₊ hydrocarbon stream 26 may then be recycled to be upgraded in the hybrid dealkylation/transalkylation unit 116 to form additional product stream 24.

As previously discussed, in some BTX-generating refinery processes, C₁₀₊ aromatic hydrocarbons are ordinarily purged from the system, such as to the fuel oil pool. One reason for doing so may be that C₁₀₊ aromatic hydrocarbons are converted less efficiently than C₉ aromatics, leading to progressive buildup of these fractions within the feed on subsequent runs. Further, C₁₀₊ aromatic hydrocarbons are known to potentially deactivate catalysts (such as by poisoning or fouling), which increasingly becomes a problem as the percentage of the feed that contains C₁₀₊ aromatic hydrocarbons increases. However, as explained in further detail in the examples infra, the hybrid transalkylation-dealkylation unit 116 and the hybrid transalkylation-dealkylation catalyst show an unexpected capability to convert C₁₀₊ hydrocarbon fractions, reducing the buildup of such fractions in the hybrid transalkylation-dealkylation unit 116 over subsequent runs. Accordingly, C₁₀₊ hydrocarbon fractions may be recycled to the hybrid transalkylation-dealkylation unit 116, which may result in utilization of the entire hydrocarbon feed towards production of BTX, and particularly para-xylenes.

Without being limited by theory, it is contemplated the unexpected capability of the hybrid transalkylation-dealkylation catalyst may be due at least in part to the partial dealkylation function of the same. Particularly, the inclusion of a dealkylation function may operate to remove alkyl groups from heavy aromatic fractions, such as the C₁₀₊ aromatics (di-ethyl benzene and/or tetra-methyl benzene, methyl-propyl-benzene, tri-methyl-ethyl-benzene, and so on, thereby forming lighter aromatic fractions such as TMB, xylenes, and toluene. As previously stated, these lighter aromatic fractions may efficiently undergo transalkylation reactions to form xylenes with the toluene feed stream 15. It is also contemplated that the dealkylation function of the hybrid transalkylation-dealkylation catalyst may, to a lesser extent, remove alkyl groups acting as bridges for bi-aromatics and multi-aromatics, such as C₁₂₊ aromatic fractions.

Still referring to FIG. 1, the p-xylene separation unit 118 may receive the xylene stream 20 as well as the additional xylene stream 20. The p-xylene separation unit 118 may also be operable separate the xylene stream 20 into a para-xylene stream 29 and a xylene isomer stream 30, such as by using adsorption, crystallization, or a combination of both, as may be understood in the art. The xylene isomer stream 30 may include m-xylene and o-xylene isomers. In embodiments, the p-xylene separation unit 118 may include one or more adsorbent columns, one or more crystallization columns, or combinations thereof.

The xylene isomerization unit 120 may receive the xylene isomer stream 30 and may isomerize the same with an isomerization catalyst to form a para-xylene rich stream 31, which may be recycled back to the p-xylene separation unit 118 for further extraction of the para-xylene stream 29 and the xylene isomer stream 30. The xylene isomerization unit 120 may be operated at an operating temperature in the range of from 200° C. to 540° C. The xylene isomerization unit 120 may also be operated at an operating pressure in the range of from 1 MPa to 5 MPa. The xylene isomerization unit 120 may also be operated with a liquid hourly space velocity of from 0.1 hr⁻¹ to 20 hr⁻¹.

The isomerization catalyst may include a mesoporous zeolite-based catalyst. The isomerization catalyst may also include a support and an active metal. The support may be selected from the group of a mesoporous mordenite zeolite, a mesoporous ZSM-5 zeolite, or beta zeolite. The active metal may be selected from the group of copper, nickel, molybdenum, tungsten, platinum, palladium, or combinations thereof. The isomerization catalyst may include from 1 wt. % to 10 wt. % mordenite by weight of the catalyst, such as approximately 4 wt. % mordenite. The isomerization catalyst may be similar to the disproportionation catalyst.

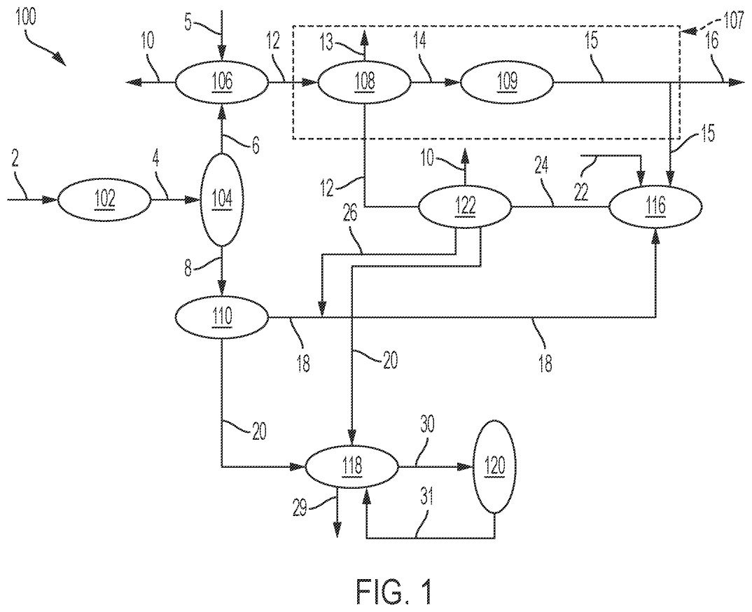

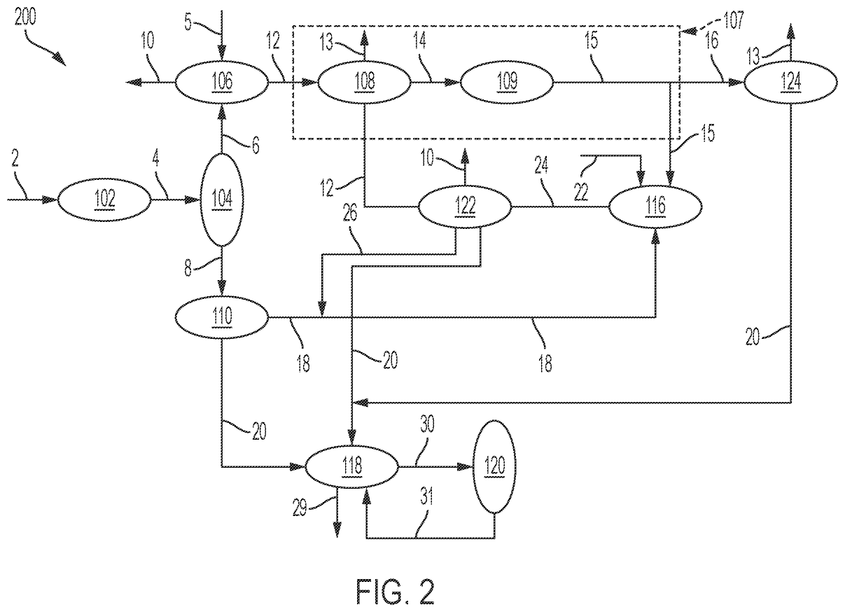

Now referring to FIG. 2, the system 200 may be similar in some or all aspects to the system 100, and may further include one or more additional treatment units or separators, as explained in further detail hereinbelow. For example, and as illustrated in FIG. 2, the system 100 may further include a toluene disproportionation unit 124 including a disproportionation catalyst. The toluene disproportionation unit may be fluidly connected to and downstream of the toluene separation unit 107, particularly the toluene column 109, and may be configured to receive at least a portion of the toluene feed stream 15 as a toluene disproportionation feed stream 16.

Without being limited by theory, the toluene disproportionation unit 124 may be included in the system to account for an excess of toluene as feed to the hybrid transalkylation/dealkylation unit 116. For example, and in embodiments, the ratio by weight of the toluene feed stream 15 to the C₉₊ hydrocarbon stream 18 may be from 0.3 to 3.0. However, as this ratio continues to increase, particularly past 1.5, the desired transalkylation rate of TMB and toluene to xylene, as well as other C₉₊ aromatic/xylene pairs, may decline, with a proportionate increase in disproportionation of toluene to xylene and benzene. Accordingly, the toluene disproportionation unit 124 may be included in the system to alleviate at least a portion of the burden of the hybrid transalkylation/dealkylation unit 116 and reduce the ratio within the hybrid transalkylation/dealkylation unit 116 to optimized levels.

The disproportionation catalyst may include a mesoporous zeolite-based catalyst. The disproportionation catalyst may also include a support and an active metal. The support may be selected from the group of a mesoporous mordenite zeolite, a mesoporous ZSM-5 zeolite, or both. The active metal may be selected from the group of copper, nickel, molybdenum, tungsten, platinum, palladium, or combinations thereof. The disproportionation catalyst may include from 1 wt. % to 10 wt. % mordenite by weight of the catalyst, such as approximately 4 wt. % mordenite. The disproportionation catalyst may be similar to the isomerization catalyst.

The toluene disproportionation unit 124 may be operated at an operating temperature in the range of from 200° C. to 540° C. The toluene disproportionation unit 124 may also be operated at an operating pressure in the range of from 1 MPa to 5 MPa. The toluene disproportionation unit 124 may also be operated with a liquid hourly space velocity of from 1 hr⁻¹ to 20 hr⁻¹.

Referring now to FIGS. 1-2, embodiments herein also include integrated processes for producing para-xylenes. The processes may include any of the integrated systems 100-200 previously described. The process may include catalytically reforming the naphtha feed stream 2 to form a reformate stream 4, separating the reformate stream 4 into the C₁-C₇ hydrocarbon stream 6 and the C₈₊ hydrocarbon stream 8, exposing the C₁-C₇ hydrocarbon stream 6 to the first solvent 5 in the solvent extraction unit 106 to form the non-aromatic hydrocarbon stream 10 and the C₆-C₇ aromatics stream 12, and separating the C₆-C₇ aromatics stream 12 to form at least the toluene feed stream 15.

The process may further include separating the C₈₊ hydrocarbon stream 8 into the C₉₊ hydrocarbon stream 18 and the xylene stream 20, separating the xylene stream 20 in the p-xylene separation unit 118 to form the para-xylene stream 29 and the xylene isomer stream 30, and isomerizing the xylene isomer stream 30 with the isomerization catalyst to produce the para-xylene rich stream 31. The process may also include upgrading the toluene feed stream 15 and the C₉₊ hydrocarbon stream 18 in the hybrid transalkylation/dealkylation unit 116 with the hydrogen stream 22 and the hybrid transalkylation/dealkylation catalyst to produce the product stream 24. In embodiments, the ratio of the toluene feed stream 15 to the C₉₊ hydrocarbon stream 18 may be from 0.3 to 3.

As previously mentioned, the additional streams produced in the hybrid transalkylation/dealkylation unit 116 may be used to enhance the production of para-xylene, such as by being recycled in one or more of the units of the systems 100-200 described herein. For example, and in embodiments, the process may further include combining the additional non-aromatic hydrocarbon stream 10 with the non-aromatic hydrocarbon stream 10; combining the additional C₆-C₇ aromatics stream 12 with the C₆-C₇ aromatics stream 12; combining the additional xylene stream 20 and the para-xylene rich stream 31 with the xylene stream 20; and combining the unconverted C₉ hydrocarbon stream and the unconverted C₁₀₊ hydrocarbon fraction stream with the C₉₊ hydrocarbon stream 18.

As illustrated in FIGS. 1 and 2, forming the toluene feed stream 15, such as in the toluene separation unit 107, may further include separating the C₆-C₇ aromatics stream 12 into a benzene-rich stream 13 and a toluene-rich stream 14, such as in the benzene column 108. Forming the toluene feed stream 15 may also include separating the toluene feed stream 15 from the toluene-rich stream 14, such as in the toluene column 109. As illustrated in FIG. 2, the process may also include sending at least a portion of the toluene feed stream 15 to the toluene disproportionation unit 124 with the disproportionation catalyst to form additional benzene-rich stream 13 and additional xylene stream 20, such as when the ratio of the toluene feed stream 15 to the C₉₊ hydrocarbon stream 18 is from greater than 1.5 to 3.

Still referring to FIGS. 1 and 2, the process may also include upgrading the unconverted C₉ hydrocarbon stream and the unconverted C₁₀₊ hydrocarbon fraction stream with the C₉₊ hydrocarbon stream 18 in the hybrid transalkylation/dealkylation unit 116 to form additional product stream 24.

### Examples

The various embodiments of processes and systems for the conversion of a naphtha feed stream into para-xylenes will be further clarified by the following examples. The examples are illustrative in nature, and should not be understood to limit the subject matter of the present disclosure.

Two pilot plant experiments were conducted according to the embodiments previously discussed and illustrated in FIG. 1. Example 1 was a base case without recycling of the unconverted C₉₊ hydrocarbon fraction 26, i.e., a single-pass. Example 2 includes recycling of the unconverted C₉₊ hydrocarbon fraction 26 to show the functionality of the hybrid transalkylation/dealkylation unit 116 to convert C₁₀₊ aromatic fractions on subsequent runs. The results of the two Examples are shown below in Table 1.

TABLE 1
Pilot Plant Experiment Results

| | Example 1 | Example 2 |
|---|---|---|
| Component | | |
| Fresh C₉₊ feed, kg/hr | 5.04 | 3.04 |
| Fresh Toluene feed, kg/hr | 3.36 | 3.36 |
| Recycle Stream | None | C₉₊ |
| Recycle feed, kg/hr | 0 | 2 |
| Hydrogen, m³/hr | 6.9 | 6.9 |
| Feed Composition | | |
| Toluene, wt. % | 39.66 | 39.92 |
| C₈, wt. % | 1.23 | 2.11 |
| MEB, wt. % | 9.82 | 7.38 |
| TMB, wt. % | 38.73 | 38.05 |
| C₉ Aromatics, wt. % | 51.42 | 48.57 |
| C₁₀₊ Aromatics, wt. % | 7.41 | 9.1 |
| Product Distribution | | |
| C₅ and below, wt. % | 5.19 | 4.27 |
| BTX, wt. % | 62.32 | 61.83 |
| Benzene, wt. % | 3.42 | 3.04 |
| Toluene, wt. % | 24.89 | 23.80 |
| Xylenes, wt. % | 34.01 | 34.98 |
| MEB, wt. % | 2.55 | 2.03 |
| TMB, wt. % | 23.14 | 24.54 |
| C₉ Aromatics, wt. % | 26.46 | 27.22 |
| C₁₀₊ Aromatics, wt. % | 5.31 | 6.14 |
| Conversions | | |
| Toluene Conversion, % | 37.26 | 40.38 |
| MEB Conversion, % | 74.01 | 72.74 |
| TMB Conversion, % | 40.24 | 35.48 |
| C₉ Conversion, % | 48.54 | 43.95 |
| C₁₀₊ Conversion, % | 28.32 | 32.24 |
| Total Conversion, % | 42.47 | 41.42 |

As shown above in Table 1, C₁₀₊ aromatics conversion increased approximately four percent after recycling the unconverted C₉₊ hydrocarbon fraction 26, indicating that the hybrid transalkylation/dealkylation unit 116 and the hybrid transalkylation/dealkylation catalyst are capable of dealkylating unconverted C₁₀₊ hydrocarbon fractions on further runs. The unconverted C₁₀₊ hydrocarbon fraction was monitored in the product stream by gas chromatography analysis of product stream samples taken every three to six hours. This gas chromatography analysis confirmed no signs of increased accumulation of C₁₀₊ hydrocarbon fractions over Example 1.

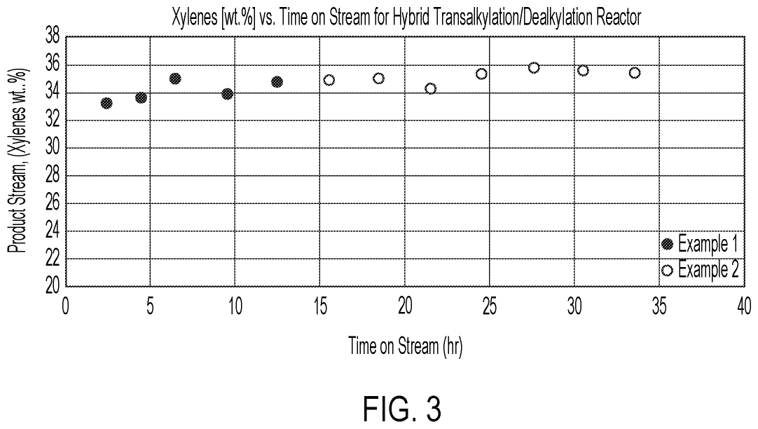

Further, as shown in Table 1 and FIG. 3, the overall BTX yield remained relatively constant between the base case (Example 1) and the subsequent run (Example 2). However, reduced yields of benzene and toluene were correlated to increased yields of xylene, the desired product. Without being limited by theory, this may also tend to show that the transalkylation and dealkylation functionalities of the catalyst may work in concert, i.e. there may be a synergistic effect. Particularly, the dealkylation functionality of the catalyst may operate to selectively dealkylate the C₁₀₊ aromatic fractions into C₉₊ aromatic fractions, such as TMB. The transalkylation functionality of the catalyst may then selectively transalkylate the toluene and in-situ generated TMB, for example, producing xylene in the preferred transalkylation reaction. It is additionally contemplated that the dealkylation functionality may also have as products toluene and mixed xylenes, also contributing to the increase xylene yield.

It is noted that recitations in the present disclosure of a component of the present disclosure being “operable” or “sufficient” in a particular way, to embody a particular property, or to function in a particular manner, are structural recitations, as opposed to recitations of intended use. More specifically, the references in the present disclosure to the manner in which a component is “operable” or “sufficient” denotes an existing physical condition of the component and, as such, is to be taken as a definite recitation of the structural characteristics of the component.

It is also noted that terms like “preferably,” “commonly,” and “typically,” when utilized herein, are not utilized to limit the scope of the claimed invention or to imply that certain features are critical, essential, or even important to the structure or function of the claimed invention. Rather, these terms are merely intended to identify particular aspects of an embodiment of the present disclosure or to emphasize alternative or additional features that may or may not be utilized in a particular embodiment of the present disclosure.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.”

Having described the subject matter of the present disclosure in detail and by reference to specific embodiments, it is noted that the various details disclosed in the present disclosure should not be taken to imply that these details relate to elements that are essential components of the various embodiments described in the present disclosure. Further, it will be apparent that modifications and variations are possible without departing from the scope of the present disclosure, including, but not limited to, embodiments defined in the appended claims.

The singular forms “a”, “an” and “the” include plural referents, unless the context clearly dictates otherwise.

Throughout this disclosure ranges are provided. It is envisioned that each discrete value encompassed by the ranges are also included. Additionally, the ranges which may be formed by each discrete value encompassed by the explicitly disclosed ranges are equally envisioned.

As used herein and in the appended claims, the words “comprise,” “has,” and “include” and all grammatical variations thereof are each intended to have an open, non-limiting meaning that does not exclude additional elements or steps.

As used herein, terms such as “first” and “second” are arbitrarily assigned and are merely intended to differentiate between two or more instances or components. It is to be understood that the words “first” and “second” serve no other purpose and are not part of the name or description of the component, nor do they necessarily define a relative location, position, or order of the component. Furthermore, it is to be understood that the mere use of the term “first” and “second” does not require that there be any “third” component, although that possibility is contemplated under the scope of the present disclosure.

What is claimed is:

1. An integrated process for producing para-xylenes, the process comprising:
catalytically reforming a naphtha feed stream to form a reformate stream;
separating the reformate stream into a C₁-C₇ hydrocarbon stream and a C₈₊ hydrocarbon stream;
exposing the C₁-C₇ hydrocarbon stream to a first solvent in a solvent extraction unit to form a non-aromatic hydrocarbon stream and a C₆-C₇ aromatics stream;
separating the C₆-C₇ aromatics stream into at least a toluene feed stream;
separating the C₈₊ hydrocarbon stream into a C₉₊ hydrocarbon stream and a xylene stream comprising ortho-xylene, meta-xylene, and para-xylene;
separating the xylene stream in a p-xylene separation unit to form the para-xylene stream and a xylene isomer stream comprising ortho-xylene and meta-xylene;
isomerizing the xylene isomer stream with a isomerization catalyst to produce a para-xylene rich stream; and
upgrading the toluene feed stream and the C₉₊ hydrocarbon stream in a hybrid dealkylation/transalkylation unit with a hydrogen stream and a hybrid transalkylation/dealkylation catalyst to produce a product stream comprising para-xylenes, wherein a ratio by weight of the toluene feed stream to the C₉₊ hydrocarbon stream is from 0.3 to 3.

2. The process of claim 1, further comprising separating the product stream into additional non-aromatic hydrocarbon stream, additional C₆-C₇ aromatics stream, an unconverted C₉ hydrocarbon fraction stream, an unconverted C₁₀₊ hydrocarbon fraction stream, and additional xylene stream.

3. The process of claim 2, further comprising:
combining the unconverted C₉ hydrocarbon stream and the unconverted C₁₀₊ hydrocarbon fraction stream with the C₉₊ hydrocarbon stream; and
upgrading the unconverted C₉ hydrocarbon fraction stream and the unconverted C₁₀₊ hydrocarbon fraction stream in the hybrid dealkylation/transalkylation unit to form additional product stream.

4. The process of claim 2, further comprising:
combining the additional C₆-C₇ aromatics stream and the C₆-C₇ aromatics stream; and
separating the combined C₆-C₇ aromatics stream into additional toluene feed stream and a benzene-rich stream.

5. The process of claim 2, further comprising:
combining the additional non-aromatic hydrocarbon stream with the non-aromatic hydrocarbon stream; and
combining the additional xylene stream and the para-xylene rich stream with the xylene stream.

6. The process of claim 1, wherein:
the hybrid transalkylation/dealkylation catalyst comprises a solid zeolite composite and an active metal;
the solid zeolite composite comprises a large pore mordenite and a medium pore ZSM-5 in a weight ratio of from 1:1 to 5:1 large pore mordenite to medium pore ZSM-5; and
the active metal is selected from the group consisting of molybdenum, chromium, platinum, nickel, palladium, rhenium, or combinations thereof.

7. The process of claim 6, wherein the active metal of the hybrid transalkylation/dealkylation catalyst is molybdenum.

8. The process of claim 6, wherein the hybrid transalkylation/dealkylation catalyst has a mesostructure comprising at least one disordered mesophase and at least one ordered mesophase.

9. The process of claim 1, wherein:
the ratio of the toluene feed stream to the C₉₊ hydrocarbon stream is from greater than 1.5 to 3; and
the process further comprises sending at least a portion of the toluene feed stream to a disproportionation unit with a disproportionation catalyst to form additional xylene stream and a benzene-rich stream.

10. The process of claim 9, wherein the disproportionation catalyst comprises:
a support selected from the group consisting of a mesoporous ZSM-5 zeolite and a mesoporous mordenite zeolite; and
an active metal selected from the group consisting of copper, nickel, molybdenum, tungsten, platinum, palladium, or combinations thereof.

11. The process of claim 1, wherein:
the reforming catalyst comprises a support and a precious metal, the support comprising silica, alumina, or silica-alumina, and the precious metal comprising platinum, ruthenium, or both;
the first solvent comprises sulfolane, n-methylpyrrolidone, di-methyl sulfoxide, n-formyl morpholine, polyglycol, or combinations thereof; and
the isomerization catalyst comprises a support selected from the group consisting of a fluorinated zeolite, a mesoporous ZSM-5 zeolite, and a mesoporous mordenite zeolite; and an active metal selected from the group consisting of copper, nickel, molybdenum, tungsten, platinum, palladium, or combinations thereof.

12. The process of claim 9, wherein the isomerization and disproportionation occur at a temperature of from 200° C. to 540° C., pressure of from 1 MPa to 5 MPa, and a liquid hourly space velocity of from 0.1 hr⁻¹ to 20 hr⁻¹.

13. The process of claim 1, wherein the hybrid transalkylation/dealkylation occurs at a temperature of from 300° C. to 480° C., a pressure of from 1 MPa to 3 MPa, a liquid hourly space velocity of from 0.1 hr⁻¹ to 10 hr⁻¹, and with a hydrogen to feed ratio of from 1 to 6.

14. An integrated system for producing para-xylenes, the system comprising:
a catalytic reformer comprising a reforming catalyst;
a first separator fluidly connected to the catalytic reformer and downstream from the catalytic reformer;
a solvent extraction unit comprising a first solvent, the solvent extraction unit fluidly connected to and downstream from the first separator;
a toluene separation unit fluidly connected to and downstream from the solvent extraction unit;
a xylene separation unit fluidly connected to and downstream from the first separator;
a hybrid transalkylation/dealkylation unit comprising a hybrid transalkylation/dealkylation catalyst, the hybrid transalkylation/dealkylation unit fluidly connected to and downstream from the toluene separation unit and the xylene separation unit;
a p-xylene separation unit fluidly connected to and downstream from the xylene separation unit; and
a xylene isomerization unit comprising a isomerization catalyst, the xylene isomerization unit fluidly connected to, downstream from, and upstream from the p-xylene separation unit.

15. The system of claim 14, wherein the toluene separation unit comprises:
a benzene column fluidly connected to and downstream from the solvent extraction unit; and
a toluene column fluidly connected to the benzene column and the hybrid transalkylation/dealkylation unit, downstream from the benzene column and upstream from the hybrid transalkylation/dealkylation unit.

16. The system of claim 15, further comprising: a sixth separator fluidly connected to the hybrid transalkylation/dealkylation unit, the benzene column, the xylene separation unit, and the p-xylene separation unit, wherein the sixth separator is downstream from the hybrid transalkylation/dealkylation unit, and upstream from the from the benzene column, the xylene separation unit, and the p-xylene separation unit.

17. The system of claim 15, further comprising a toluene disproportionation unit comprising a disproportionation catalyst, the toluene disproportionation unit fluidly connected to the toluene column unit and the p-xylene separation unit, downstream of the toluene column, and upstream of the p-xylene separation unit.

18. The system of claim 17, wherein:
the reforming catalyst comprises a support and a precious metal, the support comprising silica, alumina, or silica-alumina, and the precious metal comprising platinum, ruthenium, or both;
the first solvent comprises sulfolane, n-methylpyrrolidone, di-methyl sulfoxide, n-formyl morpholine, polyglycol, or combinations thereof;
the isomerization catalyst and the disproportionation catalyst comprise a support selected from the group consisting of a fluorinated zeolite, a mesoporous ZSM-5 zeolite, and a mesoporous mordenite zeolite; and an active metal selected from the group consisting of copper, nickel, molybdenum, tungsten, platinum, palladium, or combinations thereof; and
the hybrid transalkylation/dealkylation catalyst comprises a solid zeolite composite and an active metal, the solid zeolite composite comprising a large pore mordenite and a medium pore ZSM-5 in a weight ratio of from 1:1 to 5:1 large pore mordenite to medium pore ZSM-5, and the active metal selected from the group consisting of molybdenum, chromium, platinum, nickel, palladium, rhenium, or combinations thereof.

19. The system of claim 18, wherein the active metal of the hybrid transalkylation/dealkylation catalyst is molybdenum.

20. The system of claim 17, wherein:
the xylene isomerization unit and the toluene disproportionation unit operate at a temperature of from 200° C. to 540° C., pressure of from 1 MPa to 5 MPa, and a liquid hourly space velocity of from 0.1 hr⁻¹ to 20 hr⁻¹; and
the hybrid transalkylation/dealkylation unit operates at a temperature of from 300° C. to 480° C., a pressure of from 1 MPa to 3 MPa, a liquid hourly space velocity of from 0.1 hr⁻¹ to 10 hr⁻¹, and with a hydrogen to feed ratio of from 1 to 6.
